(19) United States
(12) Patent Application Publication
(10) Pub. No.: US 2015/0247856 A1
(43) Pub. Date: Sep. 3, 2015

(54) POTENCY ASSAY FOR SKELETAL MUSCLE DERIVED CELLS

(71) Applicant: INNOVACELL BIOTECHNOLOGIE AG, Innsbruck (AT)

(72) Inventor: Rainer Marksteiner, Schwaz (AT)

(73) Assignee: INNOVACELL BIOYECHNOLOGIE AG, Innsbruck (AT)

(21) Appl. No.: 14/430,296

(22) PCT Filed: Sep. 24, 2013
(86) PCT No.: PCT/EP2013/069818

§ 371 Date: Mar. 23, 2015

Related U.S. Application Data
(60) Provisional application No. 61/705,064, filed on Sep. 24, 2012.

(30) Foreign Application Priority Data
Sep. 24, 2012 (EP) 12185607.4

(51) Int. Cl. G01N 33/573; C12Q 1/46; A61K 35/34

(52) U.S. Cl. CPC G01N 33/573; A61K 35/34; C12Q 1/46; G01N 2500/04; G01N 2500/10; G01N 2333/916

(57) ABSTRACT

The present invention relates to a potency assay for skeletal muscle derived cells (SMDC), the potency assay comprises the steps of (a) measuring ACh E activity of SMDC, and (b) evaluating the potential of the SMDC to be used for the treatment of skeletal muscle dysfunction based on the AChE activity measured in step (a). Moreover, the present invention relates to skeletal muscle derived cells (SMDC) for use in the treatment of a muscle dysfunction. Finally, the present invention relates to the use of AChE activity as an in vitro differentiation marker for skeletal muscle derived cells and to a kit for performing the potency assay according to the present invention.

The present invention relates to a potency assay for skeletal muscle derived cells (SMDC), the potency assay comprises the steps of (a) measuring AChE activity of SMDC, and (b) evaluating the potential of the SMDC to be used for the treatment of skeletal muscle dysfunction based on the AChE activity measured in step (a). Moreover, the present invention relates to skeletal muscle derived cells (SMDC) for use in the treatment of a muscle dysfunction. Finally, the present invention relates to the use of AChE activity as an in vitro differentiation marker for skeletal muscle derived cells and to a kit for performing the potency assay according to the present invention.

Skeletal muscle derived cells comprising myoblasts are known as progenitor cells of skeletal muscles which can undergo differentiation in order to repair muscle injuries in adults.

The differentiation of mononucleated myoblasts is an essential process for muscle development and repair. Myoblast differentiation is a multi-step process involving withdrawal from the cell cycle, transcriptional activation of muscle-specific genes, and eventually cell fusion into multinucleated myotubes. The analysis of myoblast differentiation in vitro led to the knowledge that the multinucleation of myofibers only can occur through physically fusion of myoblasts. It is known that during differentiation the gene expression changes. Thus there may be many genes that become silent or activated during the differentiation of mononucleated myoblasts to multinucleated myotubes. Research on embryonic chicken myoblasts revealed that a membrane-bound acetylcholinesterase (AChE) appeared to be an early differentiation marker for skeletal myoblasts. It has been discovered that an active form of this enzyme was apparent even at mononucleated stages of myoblasts during differentiation and that no true acetylcholinesterase activity could be found on fibroblasts. In rabbit myoblasts AChE activity was described as low during the proliferation phase, increasing during myoblast fusion and decreasing during myotube degeneration. Analysis of the AChE gene and expression pattern in C2-C12 myoblasts showed that in contrast to other myogenic differentiation markers such as n-AChR subunits, the transcription rate of AChE is at the same level in myoblasts as well as in myotubes, whereas transcription rate of the n-AChR gene increases during differentiation processes. This means the increased level of AChE protein during myoblast differentiation occurs through stabilizing of mRNA transcripts. Therefore transcription of the AChE gene seems to be ubiquitous in many tissues (even in 10T1/2-fibroblast lineages), but the transcripts are degraded in most tissues, whereas transcripts are stabilized in tissues where protein expression can be detected. Reporter gene assays revealed that the 3′-UTR of AChE transcripts is a target for HuR-Proteins which increase stability and expression of AChE transcripts in differentiating C2C12 cells. Apart from the knowledge that AChE expression increases during myoblast differentiation in order to provide the function as a signal terminator at the neuromuscular junction, according to a recent study speculations were made that induction of apoptosis could lead to increased expression of AChE-R splice variants but do not affect H and T type in human myoblasts.

For example myoblasts can used in order to repair muscle injuries involved in the maintenance of continence, in particular urinary and/or anal incontinence. The loss of urinary and/or anal continence results in physical, physiological and social handicaps. Generally it is thought, that primarily elderly and handicapped people suffer from urinary and/or anal incontinence, however, these symptoms can occur in people of every age. The reasons for this can be multilayered and complex. Independently of the extremely impaired life quality for the affected individual, impaired anal continence results in a not to be underestimated cost factor for the public health system.

For using myoblasts in the treatment of muscle injuries for example for the treatment of incontinence said myoblasts are preferably isolated from a skeletal muscle biopsy of the subject to be treated. However, only fusion competent skeletal muscle derived cells are able to repair a muscle injury. There is no test quantifiable available so far for testing whether isolated muscle derived cells are indeed suitable for use in the treatment of muscle injuries. Thus, the object of the present invention is the provision of a potency assay for evaluating or verifying, whether skeletal muscle derived cells are suitable for use in the treatment of muscle injuries. In particular, the object of the present invention is the provision of a quantifiable potency assay for evaluating or verifying, whether skeletal muscle derived cells are suitable for use in the treatment of incontinence, in particular urinary and/or anal incontinence.

This object is solved by the subject matter defined in the claims.

The following figures serve to illustrate the invention.

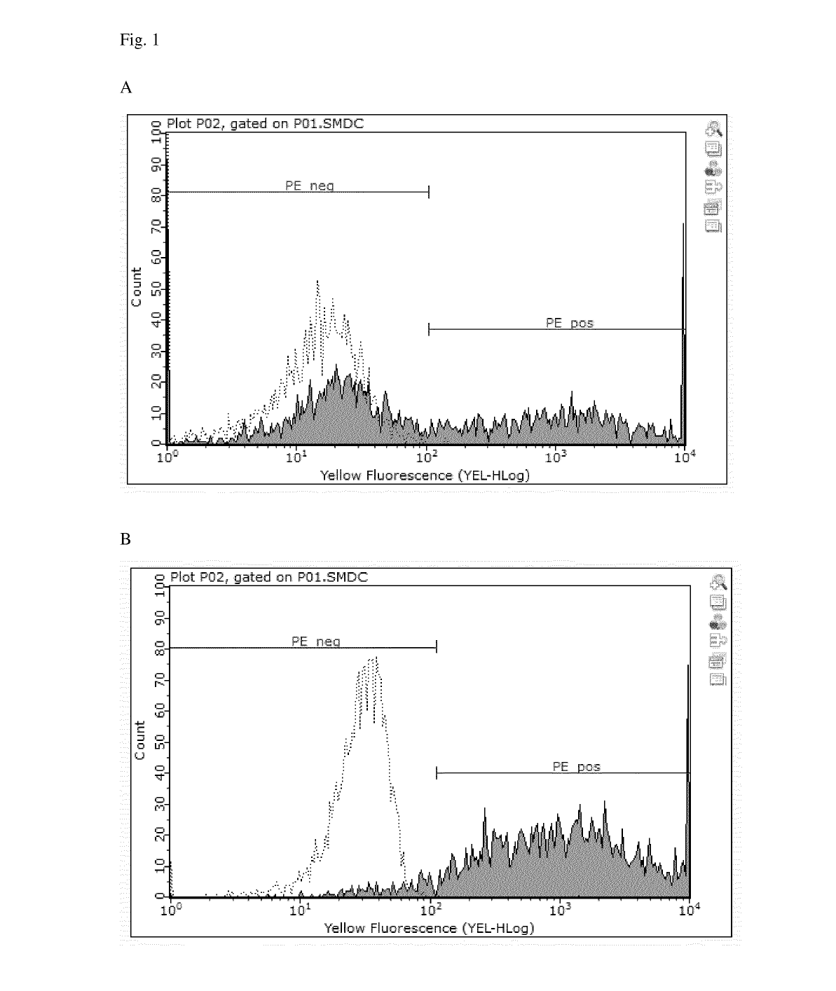

FIG. 1 illustrates CD56 expression of mixed SMDC population. 44% CD56 positive cells (A). CD56 expression of either myogenic progenitors; 92% CD56 positive cells (B) and non-myogenic progenitors; 1% CD56 positive cells (C). Red peak represents histogram of cells incubated with CD56-phycoerythrin monoclonal antibodies whereas white peak represents isotype control.

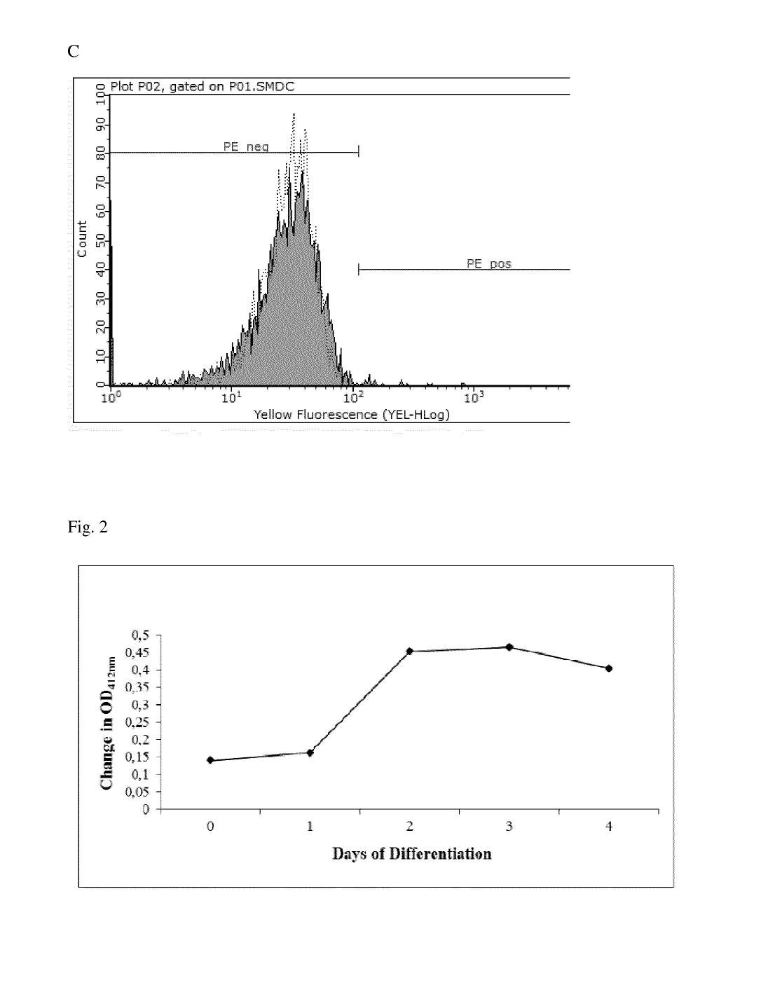

FIG. 2 shows the onset of AChE activity: Fau0113207; Changes of AChE activity during myoblast differentiation (240000 cells, CD56 positive: 95.03%). Change in OD412 nm was measured between start and 60 minutes after incubation with reagent.

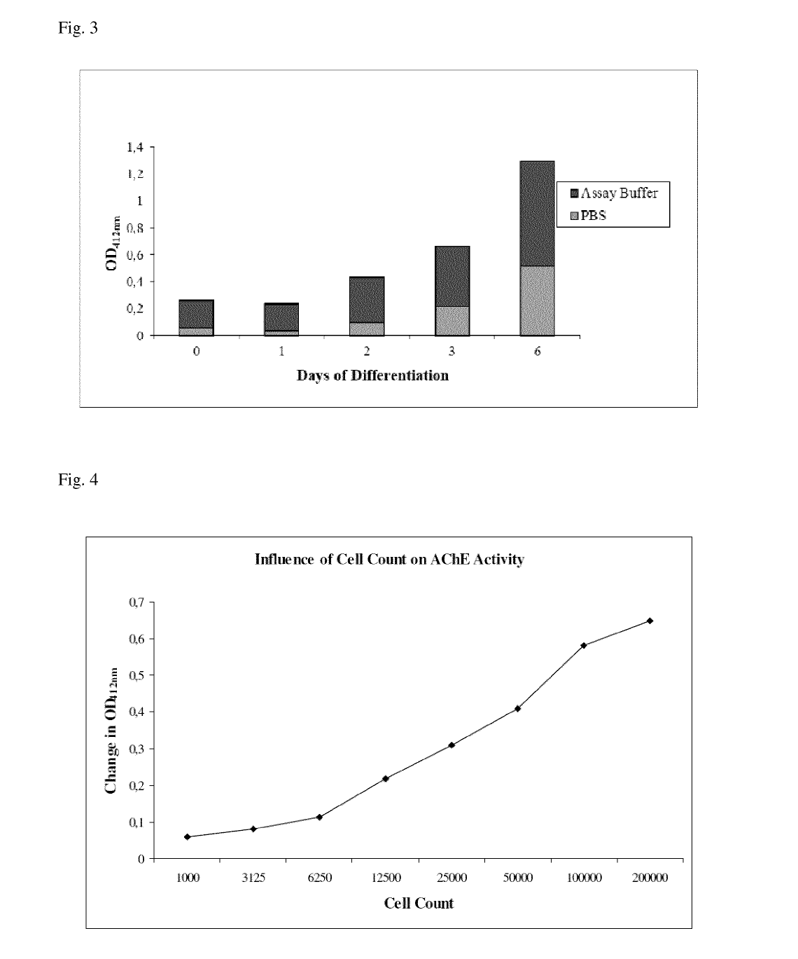

FIG. 3 shows the onset of AChE activity in Assay Buffer and in PBS: Fau0113305; OD412 nm measured after 10 min reaction time each day during differentiation within 6 days (except for day 4 and 5). 240000 cells (CD56 positive: 92, 83%) tested in each case. It is shown that membrane AChE activity (PBS buffer) increases in the same ratio then the overall AChE Activity (Assay buffer) during differentiation.

FIG. 4 shows AChE activity of different cell numbers: Fau0113305; Illustration of regression line. Different cell counts of CD56 positive SMDCs (92, 83%) have been differentiated for four days and finally AChE activity was determined. Therefore the change in OD412 nm from the start until 60 minutes after incubation was measured in a plate reader.

FIG. 5 shows AChE activity of CD56+/− cells: Fau0113166; Time drive of Acetylcholinesterase assay of mixtures (100%, 60%, 30% and 0%) of myogenic (CD56 positive) and non-myogenic (CD56 negative) cells (240000 each well) which have been differentiated for 5 days. Start points of graphs were set to zero.

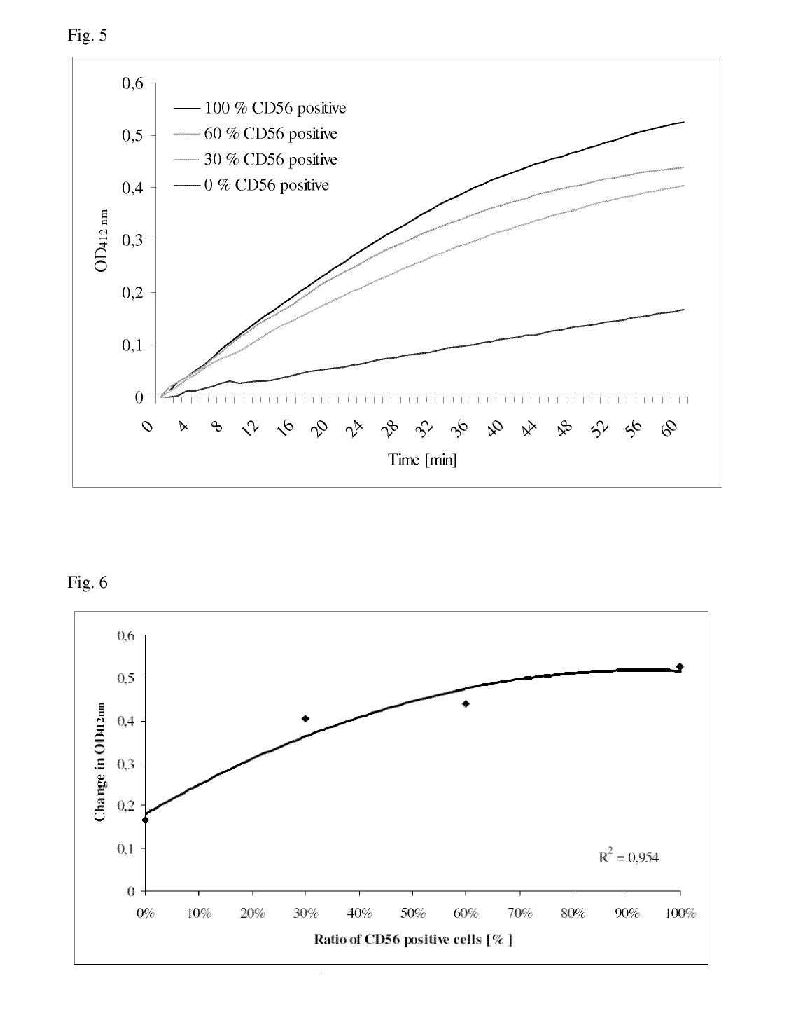

FIG. 6 shows AChE activity of CD56+/− cells: Fau0113166; Regression line between the purity of CD56 positive cells and the change in OD412 nm during 60 minutes of incubation. Mixtures of CD56 positive and CD56 negative cells (0%, 30%, 60% and 100%) of 240000 cells per well have been differentiated for 5 days.

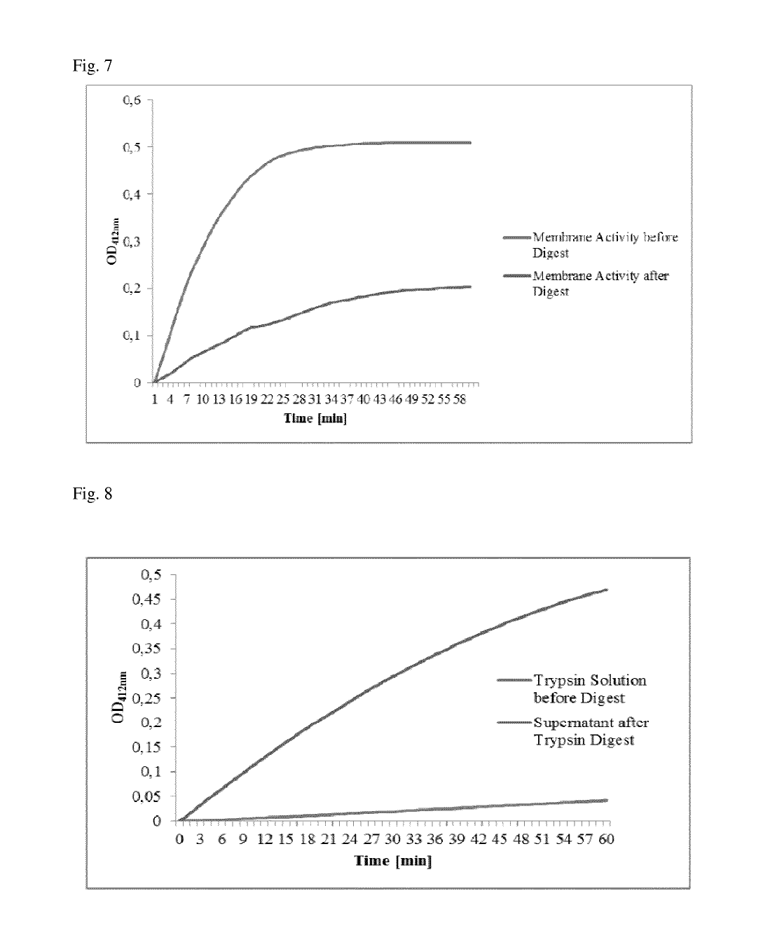

FIG. 7 shows collagenase digest of myotubes: Fau0113305; Acetylcholinesterase activity of multinucleated myoblasts (CD56 positive: >90%) after 6 days of differentiation before and after collagenase digest. Cells have been incubated with collagenase solution for 2 hours. AChE activity was measured in non-membrane permeable PBS buffer. Graphs were set to zero.

FIG. 8 shows trypsin digest of myotubes: Acetylcholinesterase activity of Trypsin solution was either tested before digestion of multinucleated myotubes (CD56 positive: >90 percentage) and after. Cells were incubated with lx trypsin solution.

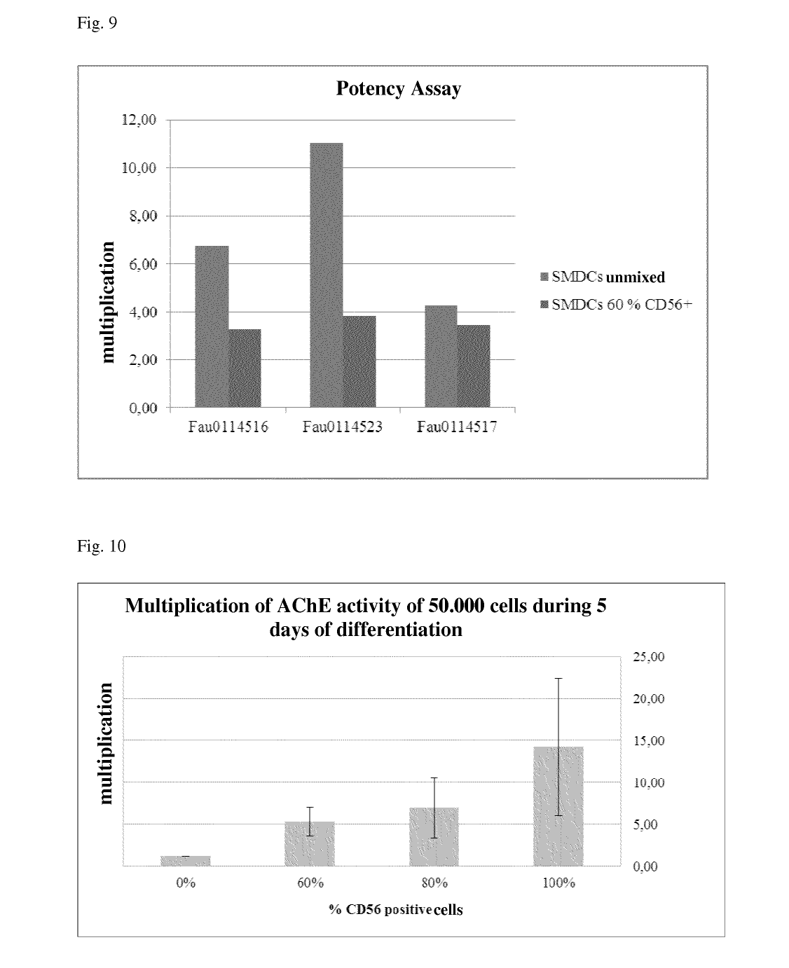

FIG. 9 shows the multiplication (0-12) of three different samples of skeletal muscle derived CD56 positive cells each compared with a mixture of cells comprising 60% CD56 positive cells.

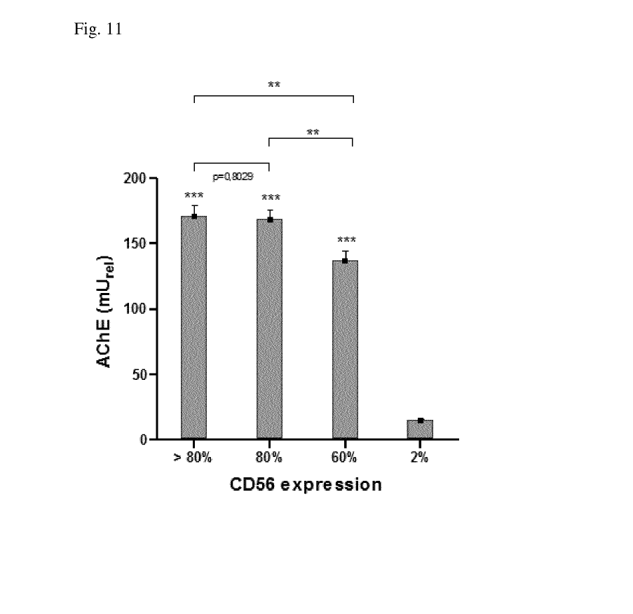

FIG. 10 shows the multiplication (0-25) of AChE activity of 50,000 cells during 5 days of differentiation. It demonstrates the linearity of the potency assay on the basis of different mixtures of CD56 positive and negative cells.

FIG. 11 shows a data analysis of an in vitro AChE assay performed on differentiated SMDCs isolated from 101 patient's muscle biopsies. AChE assay is considered positive if a test sample has ≧60 mUrel/120000 cells if at least 60% of the cells are CD56+. Data were represented as Mean±SEM and one way ANOVA was significant (**p<0.01 and ***p<0.001 vs CD56 negative cell population or among groups with different CD56%).

The use of the word “a” or “an” when used in conjunction with the term “comprising” in the claims and/or the specification may mean “one,” but it is also consistent with the meaning of “one or more,” “at least one,” and “one or more than one.”

The term “about” means that the value stated, plus or minus 5% of the stated value, or the standard error for measurements of the given value, are contemplated.

The term “acetylcholinesterase” or “AChE” as used herein refers to the enzyme Acetylcholinesterase being common in neuromuscular synapses and having the function of signal termination. It is therefore necessary for the capability of fusing myoblasts to provide interactions with neurons in the skeletal muscle. The term “AChE activity” refers to the property of cells to express AChE. Preferably, it refers to functional AChE expression during differentiation of mononucleated myoblasts to multinucleated myotubes, wherein said AChE expression is quantified. The term “AChE activity” refers preferably to the overall AChE activity and/or to membrane bound AChE activity. The “AChE activity can for example be determined by the Ellman assay (Ellman et al., Biochemical Pharmacology, 1961, Vol. 7, pp. 88-95) as well as by modified versions of the Ellman assay.

The term “potency assay” as used herein refers to a test for evaluation the quality or state of a cell population. In particular, it refers to the initial inherent capacity for development of said cell population. Preferably, the term “potency assay” as used herein refers to a quantifiable assay.

The term “urinary incontinence” as used herein, refers to any undesired loss of urine. It comprises all kinds of urinary incontinence such as stress urinary incontinence, urge urinary incontinence, mixed urinary incontinence and overflow urinary incontinence. Stress incontinence refers to urine leakage resulting after an increased abdominal pressure from laughing, sneezing, coughing, climbing stairs, or other physical stressors on the abdominal cavity and, thus, the bladder. Urge urinary incontinence is involuntary leakage accompanied by or immediately preceded by urgency. Mixed urinary incontinence refers to a combination of stress and urge incontinence.

The term “anal incontinence” or “faeces incontinence” as used herein, refers to any undesired loss of intestine content through the anus, like flatus, liquid or solid faeces. The term comprises all three severity grades: Grade 1=only gaseous, grade 2=liquid and soft feces, grade 3=solid, formed feces.

The term “urinary sphincter” or “urethral sphincter”, as used herein, refers in particular to two muscles used to control the exit of urine in the urinary bladder through the urethra. The two muscles are the external urethral sphincter and the internal urethral sphincter. The internal sphincter muscle of urethra is located at the bladder's inferior end and the urethra's proximal end at the junction of the urethra with the urinary bladder. The internal sphincter is a continuation of the detrusor muscle and is made of smooth muscle, therefore it is under involuntary or autonomic control. This is the primary muscle for prohibiting the release of urine. The external sphincter muscle of urethra (sphincter urethrae) is located at the bladder's distal inferior end in females and inferior to the prostate in males is a secondary sphincter to control the flow of urine through the urethra. Unlike the internal sphincter muscle, the external sphincter is made of skeletal muscle, therefore it is under voluntary control of the somatic nervous system. The term “urinary sphincter” or “urethral sphincter” may also refer only to the external sphincter muscle of the urethra consisting of skeletal muscle tissue.

The term “anal sphincter” or “anal sphincter apparatus,” as used herein, refers in particular to the Musculus sphincter ani externus and the Musculus puborectalis as a part of the Musculus levator ani. However it also includes M. pubococcygeus, M. ischiococcygeus, M. iliococcygeus and N. pudendus.

The term “skeletal muscle derived cell” or “SMDC” refers to multinucleated fusion competent cells as e.g. myoblasts, which can be primary cells and/or in vitro cultured cells and alternatively to other cells with myogenic potential (e.g., from liposuctioned tissue or other stem cell harbouring tissues such as bone marrow). The term also comprises cells derived from adipose which can be isolated and used for culturing of skeletal muscle cells. The term “skeletal muscle derived cell” or “SMDC” also refers to a cell population isolated from muscle tissue. Generally, such a cell population comprises further cells not having a myogenic potential. Such cells are called “non-myogenic cells” or “skeletal muscle derived non-myogenic cells” herein and are preferably CD56 negative and/or desmin negative. Thus, the term “skeletal muscle derived cell” or “SMDC” as used herein refers preferably to a cell population comprising at least 30, 40, 50, 60, 70, 80, 90, 95, 98 or 100% multinucleated fusion competent cells.

The term “penetration,” as used herein, refers to a process of introducing an injection device, for instance a needle into a body tissue without affecting the injection process yet.

The term “injection,” as used herein, refers to the expulsion of an injection solution comprising above mentioned cells out of an injection device into a specific site within the human body, in particular into or adjacent to muscle-tissue providing for urinary and/or anal continence. The injection process can be, but is not limited to, static, i.e., the injection device remains at the position reached. Alternatively, the injection process is dynamic. For instance, in some embodiments of the present invention the injection occurs simultaneously with the retraction of the injection device from the site of injection.

The term “injection site,” as used herein, refers to a site within the human body, such as close to or being muscle-tissue providing for urinary and/or anal continence, at which the injection process is initiated. The injection site needs not to be identical with the site where the injection process ends.

The term “injection device,” as used herein, refers to any device suitable for penetrating human tissue in order to reach an injection site of interest and capable of delivering solutions, in particular solutions comprising muscle-derived cells to the injection site of interest.

The term “passive incontinence,” as used herein, refers to a lack of sensory recognition of loss of urine and/or faeces.

“Imperative defecation” or “imperative urgency,” as used herein, refers to the lacking ability of a person to delay defecation for more than five minutes. Such a patient has to go to the toilette immediately.

The term “CD56+” or “CD56 positive” as used herein refers to a cell expressing the cell marker CD56. The terms “CD56+” or “CD56 positive” can also be used for a cell population comprising different cell types, if preferably at least 50, 60, 70, 80, 90, 95, 98 or 99 percent of the cell population express the cell marker CD56.

The term “CD56−” or “CD56 negative” as used herein refers to a cell not expressing the cell marker CD56. The terms “CD56−” or “CD56 negative” can also be used for a cell population comprising different cell types, if preferably at most 49, 40, 30, 20, 10, 5, 4, 3, 2, 1 or 0 percent of the cell population express the cell marker CD56.

The term “desmin positve” as used herein refers to a cell expressing the cell marker desmin. The term “desmin positive” can also be used for a cell population comprising different cell types, if preferably at least 50, 60, 70, 80, 90, 95, 98 or 99 percent of the cell population express the cell marker desmin.

The term “desmin negative” as used herein refers to a cell not expressing the cell marker desmin. The term “desmin negative” can also be used for a cell population comprising different cell types, if preferably at most 49, 40, 30, 20, 10, 5, 4, 3, 2, 1 or 0 percent of the cell population express the cell marker desmin.

The term “differentiation media” as used herein refers to cell culture media which induce fusion in multinucleated fusion competent cells or myogenic cells as e.g. myoblasts. However, said term refers also to cell culture medium not comprising any substances necessary for the induction of fusion, in case the multinucleated fusion competent cells or myogenic cells are able to fuse without a respective induction.

The term “cell growth medium” as used herein refers to any medium suitable for the incubation of mammalian cells such as SMDC, which allows the attachment of said mammalian cells on the surface of an incubation container.

The inventors of the present invention have found out that the acetylcholinesterase activity is a differentiation marker for multinucleated fusion competent cells as e.g. myoblasts that fuse to multinucleated myotubes in vitro and that there is a relation between the AChE activity and the purity of multinucleated fusion competent cells. Thus, the inventors of the present invention have surprisingly found out that the measurement of AChE activity can be used as a quantifiable test for multinucleated fusion competent cells. Furthermore, not only the overall but also the membranous AChE activity may be used as a possible marker especially because apoptotic induction leading to an increase in membrane bound activity can be excluded. The increase in AChE activity (overall or membrane bound) can therefore be described as an event depending on differentiation, cell count and the myogenic potency of skeletal muscle derived cells. Especially that only skeletal muscle derived cells with myogenic potential had an increase in AChE activity during differentiation makes the AChE activity a reliable marker for testing the myogenic potency of primary cells derived from skeletal muscles in a quantifiable test for cells with myogenic potential. Moreover, AChE activity can be used for qualifying, whether a cell population isolated from muscle tissue can be used for the treatment of skeletal muscle dysfunctions.

The present invention relates to a potency assay for skeletal muscle derived cells (SMDC). Said potency assay is a method for determining the potency of a population of skeletal muscle derived cells, the method comprising the steps of:
(a) incubating a cell population comprising skeletal muscle derived cells in a cell growth medium,
(b) incubating the cells obtained in step a) with a differentiation medium,
(c) detecting AChE activity at least two different points in time, wherein the first detection of the AChE activity is performed on the starting day of step (b), and
(d) comparing the AChE activity at the at least two different points in time, wherein the difference between the AChE activity at the at least two different points in time indicates the potency of said skeletal muscle derived cells.

The potency of a population of skeletal muscle derived cells is preferably the potency to fuse to multinucleated myotubes and/or the potency to differentiate into muscle cells. Moreover, said potency of a population of skeletal muscle derived cells may be the potency to regenerate skeletal muscle tissue. Moreover, said potency may be the potency for use of said SMDC in the treatment of skeletal muscle dysfunction such as incontinence, in particular urinary and/or anal incontinence.

The potency assay according to the present invention provides preferably a quantifiable test. That means that the potency assay according to the present invention is preferably a test for quantifying SMDC having the potency to fuse to multinucleated myotubes and/or the potency to differentiate into muscle cells.

The skeletal muscle derived cells (SMDC) are preferably cells isolated from a muscle tissue, in particular a skeletal muscle tissue and more preferably a human skeletal muscle tissue. Preferably, said skeletal muscle derived cells are multinucleated fusion competent cells or myogenic cells as e.g. myoblasts. Preferably said cells consist of at least 20%, 30, %, 40%, 50%, 60%, 70%, 80%, 90%, 95%, 99% CD56+ cells. In a specifically preferred embodiment the SMDC consist of at least 50%, 60%, 70%, 80%, 90%, 95% or 99% CD56+ cells. Alternatively or in addition said cells consist of at least 20%, 30, %, 40%, 50%, 60%, 70%, 80%, 90%, 95%, 99% desmin positive cells, more preferably of at least 50%, 60%, 70%, 80%, 90%, 95% or 99% desmin positive cells. Said skeletal muscle derived cells may be any cells obtained from a muscle tissue, in particular a skeletal muscle tissue, e.g. by a muscle biopsy. They may be a mixture of multinucleated fusion competent cells or myogenic cells such as myoblasts or sarcoblast and non multinucleated fusion competent cells such as muscle cells, fibroblasts etc. In a preferred embodiment of the present invention the skeletal muscle derived cells are purified and/or isolated. Methods for purifying and/or isolating skeletal muscle derived cells obtained from e.g. a muscle biopsy are known in the art and e.g. described in Webster et al. (Exp Cell Res. 1988 January; 174(1):252-65) or Rando et al. (J Cell Biol. 1994 June; 125(6):1275-87).

Step a) of the method according to the present invention is preferably performed under conditions suitable for the SMDC to attach to the surface of the incubation container. For example, a container coated with fibronectin can be used. Preferably, step a) is performed in a multi well plate, as e.g. in a 6-, 12-, 24-, 48- and 96 well format. For fibronectin coating, the container or multi well plate is filled with fibronectin and incubated for a sufficient time under sufficient conditions to coat the surface of said container or multi well plate with fibrotectin. For example, 100 μl Fibronectin (1-10 μg/ml) can be incubated in a 96 well plate for at least 40 minutes at a temperature from about 20° C. to about 50° C., preferably at about 37° C. Preferably, said incubation is performed in an atmosphere of about 1 to about 10% CO₂, more preferably in an atmosphere of about 5% CO₂. After said incubation the container or multi well plate is washed, preferably with PBS, preferably 1 to 5 times with 10× PBS.

The cell number used in step (a) of the method according to the present invention is preferably about 1000 to about 1×10⁶ cells, more preferably about 10000 to about 100000 cells and most preferably about 50000 cells.

The incubation of step (a) is preferably performed at a temperature from about 20° C. to about 50° C., preferably at about 37° C. Preferably, said incubation is performed in an atmosphere of about 1 to about 10% CO₂, more preferably in an atmosphere of about 5% CO₂. Said incubation is performed for a time sufficient for attaching the cells onto the surface of the container or multi well plate. Preferably, said incubation is performed for about 6 hours to about 72 hours, preferably for about 12 hours to about 36 hours and preferably for about 16 to about 24 hours.

The growth medium used in step a) of the method according to the present invention is preferably a growth medium suitable for the incubation of multinucleated fusion competent cells as e.g. myoblasts.

Preferably, the method according to the present invention comprises after step a) and before step b) a step a′) comprising removing the growth medium and adding differentiation medium. For removing the growth medium it can e.g. be discarded or aspirated. Step a′) may comprise the washing with differentiation medium. Preferably, the cells obtained in step a) are washed once with differentiation medium. After the washing with differentiation medium, fresh differentiation medium can be added for performing step b).

The incubation of step (b) is preferably performed at a temperature from about 20° C. to about 50° C., preferably at about 30° C. to about 40° C. and most preferably at about 37° C. Preferably, said incubation is performed in an atmosphere of about 1 to about 10% CO₂, more preferably in an atmosphere of about 5% CO₂. Said incubation is performed for a time sufficient for detecting an increase of AChE activity compared to the first detection of AChE activity if at least 60% CD56+ skeletal muscle derived cells are used. Preferably, said incubation is performed for about 1 day to 14 day, preferably for about 2 to about 7 days, more preferably for about 3 to about 6 days and most preferably for about 4 to about 5 days.

The first detection of AChE activity in step (c) of the present invention is performed on the same day on which the incubation of the cells obtained in step (a) with differentiation medium begins. Preferably, said detection is performed with cells obtained in step (a) which have not been contacted with differentiation medium. For said first detection the growth medium is preferably discarded or aspirated after performing step (a). Subsequently, the cells are preferably washed, preferably once with PBS. Afterwards substrate solution is added to the cells. Said substrate solution comprises preferably 1 mg substrate in 100 μl PBS. After adding said substrate solution the sample's OD is determined for detecting AChE activity. For said AChE detection the samples are preferably measured at an OD of 412 nm in a plate reader (e.g. Anthos 2010). Said detection may be performed every 60 seconds for a total of 60 minutes. Alternatively, said detection is performed in a linear area within 1 to 60 minutes, preferably after about 10 minutes, after the addition of substrate and the AChE activity is calculated based on a standard sample.

The second detection of AChE activity is preferably performed about 1 day to 14 day after the day on which the incubation of the cells obtained in step (a) with differentiation medium begins, more preferably after about 2 to about 7 days, more preferably after about 3 to about 6 days and most preferably after about 4 to about 5 days. Thus, the time difference between the two different points in time is preferably 3, 4, 5, 6 or 7 days. For said second detection the differentiation medium is preferably discarded or aspirated after performing step (b). The further procedure for said second detection can be performed as described for the first detection. Optionally, the washing step performed in the first detection can be omitted.

In step (d) of the method according to the present invention the changes in the OD is preferably calculated between 60 minutes and the beginning of the single detections. If only one AChE activity within 1 to 60 minutes is measured (no time curve but linear area) the changes in the OD is calculated in respect of the AChE activity at the at least second detection and the AChE activity at the first detection. Said change is called “OD-change”. The OD change of the first and of the second detection can then be divided for calculating the multiplication of the AChE activity within the time period between the first and the second detection.

Instead of comparing the AChE activity of SMDC after the incubation in differentiation medium with the AChE activity of SMDC prior to any incubation in differentiation medium as described in the embodiments of the potency assay as outlined above, it can be considered that the AChE activity of SMDC prior to any incubation in differentiation medium is negligible.

Thus, the present invention also refers to a potency assay for skeletal muscle derived cells (SMDC), wherein the potency assay comprises the steps of:
(a) measuring AChE activity of SMDC, and
(b) evaluating the potency of said SMDC.

Thereby, the potency of said SMDC is preferably the potency of the SMDC to fuse to multinucleated myotubes and thus the potential of the SMDC to be used for the treatment of skeletal muscle dysfunction. Thus, based on the AChE activity of SMDC in step (a) of the potency assay as outlined above the potential of SMDC to be used for the treatment of skeletal muscle dysfunction can be evaluated.

Step a) is preferably performed after SMDC have been incubated with differentiation medium. The incubation of the SMDC with differentiation medium is preferably performed under the same conditions and for the same time ranges as described above.

The SMDC subjected to the potency assay are preferably cells expressing at least the cell marker CD56. In addition, said SMDC may express further cell markers such as desmin. Thus, the potency assay may additionally comprise the step of determining, whether the SMDC express a specific cell marker such as CD56 and/or desmin. Said step is preferably performed prior to step (a) as outlined above.

When SMDC are isolated from muscle tissue the obtained cell population is usually a mixture of multinucleated fusion competent cells or myogenic cells such as myoblasts or sarcoblasts and non multinucleated fusion competent cells such as muscle cells, fibroblasts etc. Thus, alternatively or in addition the potency assay may comprise prior to step (a) as outlined above a step of enriching SMDC, which are desmin and/or CD56 positive. Methods for enriching desmin positive and/or CD56 positive cells are well known in the art. One example for a suitable enrichment method is magnetic-activated cell sorting (MACS®). The MACS method allows cells to be separated by incubating the cells with magnetic nanoparticles coated with antibodies against a particular surface antigen. Subsequently, the incubated cells are transferred on a column placed in a magnetic field. In this step, the cells which express the antigen and are therefore attached to the nanoparticles stay on the column, while other cells not expressing the antigen flow through the column. By this method, the cells can be separated positively and/or negatively with respect to the particular antigen(s). Another example for a suitable enrichment method is fluorescence-activated cell sorting (FACS®) as e.g. described in Webster et al. (Exp Cell Res. 1988 January; 174(1):252-65).

The inventors of the present invention have found out that it can be verified, whether a cell population isolated from skeletal muscle tissue and a SMDC sample, respectively, can be used in the treatment of skeletal muscle dysfunction if said cell population expresses the cell marker CD56 and/or desmin and if the AChE activity of the SMDC expressing CD56 and/or desmin is at least twice as high as the AChE activity of non-myogenic cells not expressing CD56 and/or desmin.

Thus, the potency assay of a preferred embodiment of the present invention comprises the steps of:
(a) measuring AChE activity of the SMDC, and
(b) evaluating the potential of the SMDC to be used for the treatment of skeletal muscle dysfunction, wherein the SMDC have the potential to be used for the treatment of skeletal muscle dysfunction, if the AChE activity of the SMDC are at least twice as high as the AChE activity of non-myogenic cells.

The non-myogenic cells are preferably a cell population which cannot be used in the treatment of skeletal muscle dysfunction according to the teaching of the present invention. Cells or cell populations which cannot be used in the treatment of skeletal muscle dysfunction according to the teaching of the present invention are cells which are not able to fuse to multinucleated myotubes and/or to differentiate into muscle cells. Preferably, such cells or cell population is determined by their property to express specific cell markers. The non-myogenic cells are preferably CD56 negative and/or desmin negative. Moreover, the non-myogenic cells are preferably skeletal muscle derived non-myogenic cells. For example, said skeletal muscle derived non-myogenic cells can be derived from the same subject or even from the same subject's sample as the SMDC to be tested in the potency assay. If, for example, SMDC are enriched in CD56 positive and/or desmin positive cells by magnetic-activated cell sorting (MACS®) or fluorescence-activated cell sorting (FACS®), the CD56 negative and/or desmin negative cells obtained by MACS® or FACS® may serve as non-myogenic cells.

In preferred embodiments the cell population isolated from skeletal muscle tissue have the potential to be used for the treatment of skeletal muscle dysfunction, if the AChE activity of the cell population is at least 2, 3, 4, 5, 6, 7, 8, 9, 10, 11, 12, 13, 14, 15 or 16 times higher than the AChE activity of non-myogenic cells. In a specifically preferred embodiment the AChE activity of the cell population is at least 4 times higher than the AChE activity of non-myogenic cells. Preferably, the AChE activity of the SMDC is compared with the AChE activity of non-myogenic cells measured under the same conditions.

In a preferred embodiment of the potency assays as described above the SMDC are CD56 positive and/or desmin positive. In a more preferred embodiment at least 60, 70, 80, 90, 95 or 98% of the SMDC are CD56 positive and/or desmin positive. The non-myogenic cells are preferably CD56 negative and/or desmin negative. In a preferred embodiment at least 90, 91, 92, 93, 94, 95, 96 or 97% of the non-myogenic cells are CD56 negative and/or desmin negative, most preferably at least 98%.

To verify, whether the SMDC or the non-myogenic cells are CD56 positive, desmin positive, CD56 negative and desmin negative, respectively, the expression of the cell markers CD56 and/or desmin can be verified. This verification may be incorporated in the potency assay of the present invention, preferably prior or after step (a). Alternatively, the SMDC and non-myogenic cells can be enriched on the one hand in CD56+ cells and/or desmin positive cells and on the other hand in CD56− cells and or desmin negative cells by suitable methods as e.g. magnetic cell-sorting (MACS®) or fluorescence-activated cell sorting (FACS®).

For evaluating the potential of the SMDC to be used for the treatment of skeletal muscle dysfunction it is not necessary to compare the AChE activity of CD56 positive SMDC with non-myogenic cells in each test. The evaluation of the potential of the SMDC to be used for the treatment of skeletal muscle dysfunction based on the AChE activity according to the potency assay of the present invention may also be performed based on the average AChE activity of non-myogenic cells. The average AChE activity of non-myogenic cells is easily determinable by measuring the AChE activity of a significant number of non-myogenic cells. Preferably said non-myogenic cells are derived from the same species and/or and muscle tissue from which the SMDC to be evaluated are derived. Preferably, the change of the optical density of non-myogenic cells is in the range from 0 to 0.2, more preferably in the range from 0.1 to 0.19, most preferably in the range from 0.15 and 0.18 if it is measured at 412 nm for 60 minutes of about 240000 non-myogenic cells which have been differentiated for 5 days under the conditions described in the Examples of the present invention.

The method of the present invention can be used to verify whether skeletal muscle derived cells isolated from a skeletal muscle can be used for the treatment of skeletal muscle dysfunctions. Thus, the present invention also refers to skeletal muscle derived cells (SMDC) for use in the treatment of a muscle dysfunction, wherein said SMDC have a specific AChE multiplication. Preferably, said skeletal muscle dysfunctions are a skeletal muscle dysfunction responsible for incontinence, in particular a urinary or anal incontinence. The method of the present invention can be used to verify whether skeletal muscle derived cells isolated from a skeletal muscle can be used for the treatment incontinence, in particular urinary or anal incontinence.

The inventors of the present invention have found out that skeletal muscle derived cells (SMDC) can be used for the treatment of incontinence, in particular urinary or anal incontinence, if the AChE multiplication of said SMDC corresponds at least with the AChE multiplication of a mixture comprising 60% fusion competent CD56+ cells. The remaining cells of the mixture are preferably 40% CD56− cells. Alternatively, instead of 60% CD56+ and 40% CD56− cells, 50%, 70%, 80% or 90% CD56+ cells and 50%, 30%, 20% or 10% CD56− cells, respectively, can be used. Said AChE multiplication is determined by the method according to the present invention in each case at the same test conditions. Alternatively, said MSDC can be used for the above mentioned purpose, if the AChE multiplication as detected by the method according to the present invention is at least a factor of 3, 4, 5, 6, 7, 8, 9, 10, 11, 12, 13, 14, 15, 16, 17, 18, 19 or 20. Ina preferred embodiment the SMDC, which are suitable for use in the treatment of a muscle dysfunction, exhibit an AChE multiplication of at least four in differentiation medium. Preferably, said factor is obtained if the second detection is performed 5 days after the first detection. As a further alternative, said SMDC can be used for the above mentioned purpose, if the AChE activity of said SMDC is at least twice as high as the AChE activity of non-myogenic cells. In a more preferred embodiment said SMDC can be used for the above mentioned purpose, if the SMDC comprise at least 60% CD56 positive and/or desmin positive cells and the AChE activity of said SMDC is at least twice as high as the AChE activity of non-myogenic cells. In a more preferred embodiments, the SMDC comprise at least 70, 80, 90, 95, 98% CD56 positive and/or desmin positive cells. Alternatively or in addition the AChE activity of the SMDC is at least 2, 3, 4, 5, 6, 7, 8, 9, 10, 11, 12, 13, 14, 15 or 16 times higher than the AChE activity of non-myogenic cells.

The MSDC for use in the treatment of incontinence are preferably homologous to the recipient. In a more preferred embodiment said MSDC are autologous or heterologous to the recipient. Said MSDC may e.g. be obtained by a biopsy of the biceps of the recipient. Autologous MSDC reduce or minimize the risk of allergic reactions, after the MSDC have been injected into the recipient. Preferably, the MSDC are multinucleated fusion competent cells or myogenic cells such as myoblasts. More preferably, the said MSDC are human cells.

Myoblasts, the precursors of muscle fibers, are mononucleated muscle cells which differ in many ways from other types of cells. Myoblasts naturally fuse to form post-mitotic multinucleated myotubes which result in the long-term expression and delivery of bioactive proteins. Myoblasts have been used for gene delivery to muscle for muscle-related diseases, such as Duchenne muscular dystrophy, as well as for non-muscle-related diseases, e.g., gene delivery of human adenosine deaminase for the adenosine deaminase deficiency syndrome; gene transfer of human proinsulin for diabetes mellitus; gene transfer for expression of tyrosine hydroxylase for Parkinson's disease; transfer and expression of Factor IX for hemophilia B, delivery of human growth hormone for growth retardation.

In view of the provision of the potency assay according to the present invention, the present invention provides more effective methods for the prevention or treatment of urinary and/or anal incontinence, by delivering MSDC to muscle tissues of the urinary tract, to the urinary sphincter system, rectum, to the anal sphincter system, and/or to the external anal sphincter. Therefore, the present invention relates to methods of preventing or treating urinary and/or anal incontinence, wherein the method comprises the following steps: (a) verifying the potency of previously obtained MSDC by the potency assay according to the present invention, (b) introducing of an injection device through the skin or urethra of a patient, (c) moving the injection device forward until the injection device reaches the injection site of interest, and (d) injecting of said previously obtained and verified MSDC via said injection device into said injection site of interest, wherein the injection site of interest is, or is adjacent to, muscle-tissue providing for urinary and/or anal continence. Step (d) may further comprise withdrawing the injection device from the site of interest while, at the same time, said muscle-derived cells are dispensed from said injection device along a least a portion of the injection canal created by the moving of said injection device into the injection site of interest, thereby creating an injection band. In one embodiment the injection band may be no more than about 600 μm in diameter and/or the length of the injection band may be as long as the muscle being injected.

In a particular embodiment, the muscle-tissue providing for urinary and/or anal continence is the anal sphincter system, the internal anal sphincter, and the external anal sphincter. In a further embodiment, the muscle-tissue for anal continence is M. puborectalis. The muscle-tissue providing for urinary continence is preferably the urinary sphincter system, the internal urinary sphincter and the external urinary sphincter.

Additionally, the present invention relates to methods of preventing or treating urinary and/or anal incontinence, wherein the method comprises the following steps: (a) verifying the potency of previously obtained MSDC by the potency assay according to the present invention, (b) introduction of an injection device into the rectum and/or urinary tract of a patient, (c) moving the injection device forward along the rectum until the injection device reaches the plane of the injection site of interest; (d) penetrating the urethral wall and/or the area between the skin and the rectum wall with the injection device; and (e) moving the injection device forward until the injection device reaches the injection site of interest, and subsequently, (f) injecting of previously obtained muscle-derived cells via the injection device into the injection site of interest, wherein the injection site of interest is, or is adjacent to, muscle-tissue providing for urinary and/or anal continence. Step (f) may further comprise withdrawing the injection device from the site of interest while, at the same time, said muscle-derived cells are dispensed from said injection device along a least a portion of the injection canal created by the moving of said injection device into the injection site of interest, thereby creating an injection band. In one embodiment the injection band may be no more than about 600 μm in diameter and/or the length of the injection band may be as long as the muscle being injected.

In one embodiment the skeletal muscle-derived cells to be injected can be autologous skeletal muscle-derived cells (e.g., myoblasts, and muscle-derived stem cells (MDCs)). When practicing the present invention these cell types may be injected into or adjacent to an injured muscle tissue providing for urinary and/or anal continence, e.g., an injured anal sphincter externus as means of prevention or treatment for anal incontinence or in the urinary sphincter as means of prevention or treatment for urinary incontinence.

The previously obtained skeletal muscle-derived cells, i.e., obtained prior to practicing the methods of the present invention, can be cultured cells which can generate sufficient quantities of muscle cells for repeated injections. Alternatively, the skeletal muscle-derived cells are primary cells.

Therefore, the present invention also provides a simple prophylaxis approach or treatment method for women and men with urinary and/or anal incontinence or in risk of developing urinary and/or anal incontinence by using autologous muscle-derived cells to enhance their urinary and/or anal sphincters. Such muscle-derived cell therapy allows repair and improvement of damaged urinary and anal sphincter. In accordance with the present invention the treatment comprises a needle aspiration to obtain muscle-derived cells, for example, and a brief follow-up treatment to inject cultured and prepared cells into the patient. Also according to the present invention, autologous muscle cell injections using myoblasts and muscle-derived stem cells (MDCs) harvested from and cultured for a specific urinary and/or anal incontinence patient can be employed as a non-allergenic agent to bulk up the urinary and/or rectum wall, thereby enhancing coaptation and improving the urinary and/or anal sphincter muscle. In this aspect of the invention, simple autologous muscle cell transplantation is performed, as discussed above.

It is contemplated that any method or composition described herein can be implemented with respect to any other method or composition described herein.

In accordance with the present invention, skeletal muscle-derived cells, including myoblasts, may be primary cells or cultured cells. They may be histocompatible (autologous) or nonhistocompatible (allogeneic) to the recipient, including humans. Particular embodiments of the present invention are myoblasts and muscle-derived stem cells, including autologous myoblasts and muscle-derived stem cells which will not be recognized as foreign to the recipient. In this regard, the myoblasts can be matched vis-á-vis the major histocompatibility locus (MHC or HLA in humans). Such MHC or HLA matched cells may be autologous. Alternatively, the cells may be from a person having the same or a similar MHC or HLA antigen profile. The patient may also be tolerated to the allogeneic MHC antigens. The present invention also encompasses the use of cells lacking MHC Class I and/or II antigens, such as described in U.S. Pat. No. 5,538,722.

Establishment of a primary skeletal muscle-derived cell culture from isolated cells of muscle tissue can be obtained by methods well known to a person skilled in the art, e.g., via a muscle biopsy. Such muscle biopsy serving as the source of skeletal muscle-derived cells can be obtained from the muscle at the site of injury or from another area that may be more easily accessible to the clinical surgeon. As mentioned above, the skeletal muscle-derived cells need not necessarily to be obtained from the patient to be treated. However, an embodiment of the invention is where the muscle biopsy is taken from the patient suffering from urinary and/or anal incontinence. The site of the biopsy is not restricted but may be a skeletal muscle, such as from the upper arm. A biopsy of the biceps is especially preferred. The size of the biopsy may comprise approximately 1 cm×1 cm×1 cm or bigger. From this biopsy sample satellite cells, i.e., cells capable to fuse (syncytium of at least three cells) and to establish an oriented, contractile cytoskeleton (actin-myosin sequence) are isolated and cultured. About 60 to about 500 million cells may be cultured for a single treatment. Additionally, a blood sample can be obtained from the patient, which is subsequently used for cultivation of the cells in vitro. Alternatively, fetal bovine serum is used for cultivation. Myoblasts in cell culture can be further purified using an established technology (Rando and Blau, 1994) or other methods. These muscle cells are cultivated in vitro.

In a muscle biopsy, a small area of muscle tissue generally contains enough myogenic cells to produce millions of skeletal muscle-derived cells in culture. For myoblasts, once the cells are isolated and grown in culture, it is easy to distinguish pure myoblasts from other cell types, since myoblasts fuse to form elongated myotubes in vitro.

The MSDC, which can be used for the treatment of a muscle dysfunction, in particular for the treatment of incontinence such as urinary and/or anal incontinence, exhibit preferably a characteristic expression pattern. Preferably, more than about 60%, 70%, 80%, 90%, 95% or 98% of said MSDC express CD56 and/or desmin. Preferably, said MSDC do not express CD34, Sca-1 and MyoD. Thereby, the term “do not express” means that preferably less than 40%, 30%, 20%, 10%, 5% or 2% of the MSDC express said markers. The expression pattern of MSDC as described above can be used to determine the myogenicity index of the cell culture without the requirement of differentiation. Thus, said expression pattern of MSDC can be used either in addition to the potency assay of the present invention or instead the potency assay of the present invention to verify, whether skeletal muscle derived cells can be used for the treatment of a muscle dysfunction, in particular for the treatment of incontinence such as urinary and/or anal incontinence.

In general, injecting skeletal muscle-derived cells, including myoblasts, skeletal muscle-derived stem cells or other cells with myogenic potential (see above), into a given tissue or site of injury comprises a therapeutically effective amount of cells in solution or suspension, e.g., about 1×10⁵ to about 6×10⁶ cells per 100 μl of injection solution. In particular, for the treatment of urinary incontinence an amount of 100,000 to 300,000 cells, more preferably 200,000 is preferred. For the treatment of anal incontinence a higher amount of cells is preferred. The injection solution is a physiologically acceptable medium, with or without autologous serum. Physiological acceptable medium can be by way of non-limiting example physiological saline or a phosphate buffered solution.

In one embodiment of the present invention, skeletal muscle-derived cell injection, autologous myoblast injection, into the external anal sphincter and urinary sphincter, respectively, is employed as a treatment for anal incontinence and urinary incontinence, respectively to enhance, improve, and/or repair the sphincter. Skeletal muscle-derived cells, such as myoblasts, are injected into the sphincter and survive and differentiate into myofibers to improve sphincter function. The feasibility and survival of myoblast injection into the external anal sphincter has been verified. In accordance with this embodiment, autologous skeletal muscle-derived cell injections (i.e., skeletal muscle-derived cells harvested from and cultured for a specific incontinence patient) can be used as a non-allergenic agent to bulk up the rectum wall and/or urethral wall, thereby enhancing coaptation and improving the sphincter muscle function by integration into the striated muscle fibres of the muscle.

In accordance with the present invention autologous skeletal muscle-derived cells administered directly into the urinary sphincter and/or anal sphincter exhibit long-term survival. Thus, autologous myoblast injection results in safe and non-immunogenic long-term survival of myofibers in the urinary and/or anal sphincter.

In a particular embodiment according to the invention, about 50 to about 200 μl of a skeletal muscle-derived cell suspension (with a concentration of about 1×10⁵ to about 6×10⁶ cells per 100 μl of injection solution) are injected into the urinary sphincter. The injection device can be connected to a container containing the cell suspension to be injected. For the treatment of anal incontinence preferably about 50 μl to about 1 ml, more preferably about 0.5 ml of a skeletal muscle-derived cell suspension (with a concentration of about 1×10⁵ to about 6×10⁶ cells) are injected into the external anal sphincter or into the urinary sphincter. The injection device can be connected to a container containing the cell suspension to be injected.

In another embodiment, the injection step may comprise several individual injections, such as about 20 to about 40 injections of skeletal muscle-derived cell suspension, wherein in each injection about 50 to about 200 μl of a skeletal muscle-derived cell suspension are injected and wherein each injection is applied to another region of the anal sphincter. However, these parameters have to be considered as being merely exemplarily and the skilled artisan will readily be able to adapt these procedures to the treatment requirements for each individual patient.

In another embodiment of the present invention, the movement of the injection device towards the urinary and/or anal sphincter is monitored by sonography and/or EMG (electromyography) means. In a particular embodiment, a transrectal probe is introduced and the position of the transrectal probe is adjusted optimally for the treatment of the urinary and/or anal sphincter with the methods according to the invention. In another particular embodiment, the skeletal muscle-derived cells are implanted in the area surrounding the urinary and/or anal sphincter defect and/or especially in the area of the urinary and/or anal sphincter defect. The patient can start the next day after injection of cells with physical exercises to further the treatment of urinary and/or anal incontinence according to the invention.

In another embodiment, the treatment is repeated. The treatment can be repeated e.g. within one year after the last treatment, after 10, 9, 8, 7, 6, 5, 4, 3, 2 or 1 month(s) after the last treatment or within 1 to 8 weeks, preferably 2 to 3 weeks, or 10 to 20 days after the last treatment. In particular, the treatment can be repeated within 2 to 3 weeks after the last treatment with cells from the very same cell culture as used for the prior treatment. This approach allows for a reduced injection volume per injection and gives the cells more time to adapt and to integrate and to build up the muscle. In an even more specific embodiment, the injections are repeated in time intervals of 2 to 3 weeks until an improvement of urinary and/or anal continence is achieved.

As mentioned above, a particular penetration route is through the skin of a patient in parallel to the course of the rectum. However, it is also contemplated, that the penetration can occur directly from the rectum in the vicinity of the injured muscle. In particular, the penetration and injection process is monitored via sonographic imaging means. Additionally, an alternative penetration route is contemplated for women, that is, trans-vaginal injection. In this scenario, the injection device penetrates the wall of the vagina and is moved forward until it reaches the desired injection site. In particular, the penetration and injection process is monitored by sonographic and/or EMG (electromyography) imaging means in this scenario as well.

In another embodiment, the injection comprises injecting the skeletal muscle-derived cells in form of an “injection band.” “Injection band,” as used herein, refers to disposition of cells along the length, or a portion of the length, of the injection track, i.e., along the canal created by insertion of the needle into the muscle tissue. In other words, following injection, the needle is withdrawn while, at the same time, cells are expelled from the syringe in a continuous or intermittent fashion with the injection needle is moved, in particular, retracted along the injection track. Such steady dispensing of cells provides for a continuous delivery of the injection solution, including cells, along the injection canal that is formed when the injection device/needle enters the target muscle-tissue. In a particular embodiment, the injection band or canal should have a diameter not bigger than about 600 μm, since this would lead to necrosis of the skeletal muscle-derived cells in the center of the injection canal, and consequently, result in detrimental inflammation and other processes.

The injection device for use with the methods of the present invention may be any device capable of penetrating human tissue and capable of delivering solutions, in particular solutions comprising skeletal muscle-derived cells to a desired location within the organism of a subject, in particular of a human subject. The injection device can comprise, for instance, a hollow needle. The injection device may also be any type of syringe suitable for injecting skeletal muscle-derived cells. In more sophisticated embodiments, the injection device can be for example an injection gun, injecting the cell suspension by applying air pressure. In particular, the injection device is suited for keyhole applications and keyhole surgery, respectively.

By choosing an injection needle having a particular diameter, the injection volume per mm³ can be exactly pre-determined. The diameter of the injection needle will normally not exceed 5 mm, as this can lead to damage of the muscle structures.

Sonographic imaging means for monitoring the position and action of the injection device can be achieved by any standard ultrasonic imaging device known in the art. In addition to mono- or biplanar standard ultrasonic probes, also new ultrasonic technologies can be used, such as, for example, 3D-sonography or color Doppler sonography, etc. In a particular embodiment, as discussed above, the injection device comprises a sonographic imaging means.

A further aspect of the present invention is the use of AChE activity as an in vitro differentiation marker for skeletal muscle derived cells.

Finally, a further aspect of the present invention is a kit comprising means for performing the potency assay according to the present invention. Said kit may for example comprise multi well plates, growth medium, differentiation medium and/or a substrate solution for performing the potency assay according to the present invention. Said kit is preferably a kit for quantifying SMDC having the potency to fuse to multinucleated myotubes and/or the potency to differentiate into muscle cells. More preferably said kit is a kit for evaluating the potential of the SMDC to be used for the treatment of skeletal muscle dysfunction, in particular of urinary and/or anal incontinence.

The following examples explain the present invention but are not considered to be limiting.

### EXAMPLE 1

### Isolation of Human SMDC Comprising Fusion Competent Cells (Myoblasts)

SMDC were isolated from a patient's biceps biopsy. The muscle biopsy was placed in a sterile petri dish. A few drops of PBS were added and the muscle was minced into a slurry by razor blades. Then, cells were enzymatically dissociated by the addition of 2 ml per g of tissue of a solution of dispase (grade II, 2.4 U/ml, Roche Applied Science) and collagenase (class II, 1%), supplemented with CaCl₂ to a final concentration of 2.5 mM. After maintaining the slurry at 37° C. for 30-45 min it was triturated every 15 min with a 5-ml plastic pipette and then passed through 80 μm nylon mesh (Nitex; Tetko). The filtrate was spun at 350 g to sediment the dissociated cells. The pellet was resuspended in growth medium and the suspension was plated on collagen-coated dishes. During the first several passages of the primary cultures, fusion competent cells were enriched by preplating as e.g. described in Richler et al. (Dev. Biol, 1970, 23:1-22).

### EXAMPLE 2

### Cultivation of Adherent Primary Cell Culture

SMDC comprising myoblasts were cultivated in Ham's F10 basal medium, which was supplemented with 10% FCS, bFGF and gentamycin. A sterilfiltration trough a 0.2 μm filter was carried out. Cells were seeded on standard culture flasks for proliferation and medium was changed every three days. For subcultivation and harvest, the cells were washed once with PBS and incubated with Trypsin solution (diluted 1:10 in PBS) for 5 min in an incubator (37° C., 5% CO2). Cells were then rinsed with culture medium and centrifuged in an expendable tube at 1300 rpm (400 rcf) for ten minutes, supernatant discarded and pellet resuspended in growth medium.

### EXAMPLE 3

### Cell Count

Counting of cells was carried out according to the manual of chemometec nucleocounter™. This method uses Propidiumiodid staining of nuclei and calculates the number of cells in 0.2 ml. Before automatic counting, 100 μl cell suspension was mixed with 200 μl of Reagent A—Lysis Buffer (in order to permeabilize the cell membrane) and incubated at room temperature for 5 minutes. Then 200 μl of Reagent B—Stabilizing Buffer was added. The suspension was mixed, collected with a nucleocasette and finally measured.

### EXAMPLE 4

### Cryopreservation of Cells

Cells were preserved cryogenically as following: Cells were first harvested and centrifuged at 1300 rpm (400 rcf) 10 min, supernatant was discarded and pellet resuspended in 1 ml Cryomaxx-Medium I™ per 1 million cells. The suspension was finally transferred to cryovials, froze to −140° C. with the ICE-Cube™ and stored in a liquid nitrogen filled cryotank. For recultivation of cryopreservated cells frozen tubes were put into a water bath (37° C.) for thawing. Afterwards cell suspension was diluted 1:30 with warmed (37° C.) growth medium and seeded on a culture flask.

### EXAMPLE 5

### Cell Differentiation

Differentiation of human myoblasts to syncitial myotubes takes place when growth medium is replaced by a serum-free differentiation medium. Skeletal Muscle Cell Differentiation Medium (catalogue nr. 23061, Promo Cell) was supplemented with a Skeletal Muscle Cell Differentiation Medium Supplement Pack (as described in protocol of company, from which Medium has been obtained) and 250 μl of gentamycin. For the onset of differentiation, cells (in growth medium) were seeded in 4-well or 24-well dishes (60000-480000 cells). After the cells were attached to the dish (overnight), growth media was discarded, cells were washed once with differentiation medium and covered with 500 μl differentiation medium.

### EXAMPLE 6

### FACS Analysis

For FACS-analysis, cells had to be harvested (as described in Example 2) and number of cells per ml determined (described in Example 3). 50000 cells were put into a vial and filled up to a total amount of 600 μl with growth medium. 40 μl of 7AAD viability dye was added to the cell suspension. Then 20 μl of each antibody was filled into separate tubes, which afterwards where filled with 300 μl of the suspension mixed up before. The tubes where then incubated in the dark for 15 minutes. After mixing the suspensions well fluorescence could be measured. For phycoerythrin-conjugated antibodies FL1 (yellow-fluorescence) channel was activated and for 7AAD viability-dye the FL4 (red-fluorescence) channel was activated. A standard protocol for all FACS measurements had been created in advance, where all parameters were defined as well as compensation of phycoerithrin and 7AAD was carried out.

### EXAMPLE 7

### Magnetic-Activated Cell Sorting

Magnetic-activated cell sorting is a method for purifying cells in dependence of one (or more) cell surface antigen(s). For this method antibodies which had been conjugated to magnetic mircobeads before were used. Experiments were carried out almost equally to the protocol of MACS® company of which CD56 antibodies were obtained and preceded as follows. After harvesting of cells (as described in Example 2) and measuring of entire cell number (Example 3) cells were centrifuged again at 1300 rpm (400 rcf) for ten minutes, supernatant discarded and resuspended in 10 ml MACS-Buffer. After another centrifugation step (1300 rpm, 10 min) and discarding of supernatant, the pellet was resupended in 80 μl MACS-Buffer each 107 cells but not less than 80 μl. Subsequently 20 μl of magnetic CD56 antibody was added per 107 cells and incubated for 15 minutes at 4° C. Afterwards sorting of cells was carried out as described in MACS protocol with Mini MACS Separator and MS-column.

### EXAMPLE 8

### Indirect Immunostaining with Fluorescent-Conjugated Secondary Antibodies

Indirect immunofluorescence staining was conducted only on adherent cells. Cells were washed two times with 500 μl PBS and afterwards incubated with 500 μl of 2% (v/v) formaldehyde (diluted in PBS) for 20 minutes at room temperature. After washing twice with 500 μl PBS, cells were covered with 500 μl antibodies. The used primary antibodies (AChE or AChR) were diluted in advance to a final concentration of 40 μg pro ml (w/v) with PBS. The covered cells were then incubated for at least 90 minutes (37° C., 5% CO2). Followed by another washing step (as described above), cells were covered with 500 μl secondary antibodies (40 μg/ml) and incubated for 60 minutes at 37° C. and 5% CO2. Afterwards cells were washed three times with 500 μl PBS and could then be analysed through a fluorescence microscope.

Indirect Immunostaining with Biotin-Conjugated Secondary Antibodies

First supernatant of cell culture dishes was discarded and cells washed three times with PBS. Permeabilisation and fixation was carried out by covering the cells with 2% formaldehyde solution (v/v; diluted in PBS) for 20 minutes at room temperature. Then the cells were washed with PBS three times after each conducted incubation step. Afterwards cells were covered with 500 μl hydrogenperoxid-block and have been incubated for five minutes at room temperature. Primary antibodies (AChE or desmin) in a final concentration of 40 μg pro ml (w/v) were pipetted on the cells and have been incubated for at least 90 minutes (37° C., 5% CO2). Cells were then covered with 500 μl biotin-conjugated secondary antibodies (goat anti rabbit, polyclonal) and have been incubated same as for primary antibodies but 60 minutes at least. For visualisation of antibody bindings, 500 μl of horseradish streptavidin peroxidase had to be added in a final concentration of 2-5 μg/ml (diluted in PBS) and incubated at 37° C., 5% CO2, 20 minutes long followed by covering the cells with 500 μl chromogen single-solution, which was removed after 5 to 15 minutes. A final washing step with PBS was carried out before results could be observed.

### EXAMPLE 9

### Quantitative Analysis of AChE Enzyme Activity

Quantitative measuring of AChE-activity was carried out by using an acetylcholinesterase assay kit (Quantichrom Acetylcholinesterase Assay), which is based on an improved Ellman assay (Ellman, G. L., Biochem. Pharmacol., 7, 88-95, 1961). Therefore, instructions on the manual were considered but changed for improving the results.

Preparation of reagent: 5, 5′-dithiobis (2-ntrobenzoic acid) and acetylthiocholine iodide was either diluted in PBS or Assay Buffer by adding 200 μl of chosen buffer to 2 mg substrate. Measurement preferences of Spectrometer:

The following attributes are these, which were the same for every spectrometric measurement and accord to the used software, UV-Winlab.

Overall preferences:

Ordinate max: two

Ordinate min: zero

Lamp Visible: on

Response time: 0, 5 sec

Slit width: 1, 0 nm

Lamp change: 326 nm

Measured extinction: 412 nm

Cell changer: on

Preferences according to time drive experiments:

Time interval: 60 sec

Display: serial

Preparation of cells: Two ways of cell preparations were performed as described following.

Measuring of Cells by Detaching from Surface

Supernatant of cells cultivated in 4-well culture dishes was discarded first and cells covered with 200 μl Trypsin. After 5 minutes of incubation at 37° C. and 5% CO2 cells were rinsed with PBS and put into a 15 ml Falcon Tube. Next, the cells have been centrifuged at 800 g for 10 minutes. Supernatant was discarded and 300 μl of the Reagent (diluted in either PBS or Assay Buffer) were added. Cells were then mixed briefly and transferred to a cuvette of appropriate size. Optical density (OD) could then be measured as a time drive on a Spectrometer. For each measurement a cuvette filled with 300 μl of distilled sterile H₂O as well as one cuvette with Calibrator were measured also. If needed, depending on experimental conditions, a blank value was measured as well. Finally, AChE activity in unit per litre [U/L] could be calculated as described in product description: AChE Activity=(OD10−OD2)/(ODCAL−ODH2O)*200. Because this formula was made for AChE activity measurement of blood and other soluble samples, it was not used for our experimentation. However, AChE activity was determined by the change in absorption of light at the wavelength of 412 nm.

Measuring of Cells without Detaching from Surface

After discarding of supernatant, 300 μl of preliminary prepared Reagent-Buffer mix was added to the cells (cultivated in 4-well or 24-well culture dishes). The cells then have been incubated for a time (dependent on the time relevant for experiment) at 37° C. and 5% CO2. Afterwards supernatant was transferred into a cuvette and OD measured on a Spectrometer. OD at 412 nm therefore stands in direct correlation to the amount of active AChE in the cells. For measuring of AChE activity with a Plate Reader (Anthos 2010), cells were seeded in myoblast growth media on flat bottom 96-well culture dishes and incubated at 37° C., 5% CO2. Growth media was replaced the next day by skeletal muscle cell differentiation media. Further incubation as described above. After several days of differentiation, differentiation media was replaced and cells washed with PBS once. AChE assay reagent has been prepared as described before and 100 μl put on each well. OD412 nm then has been measured for 60 minutes on an Anthos Plate Reader through a kinetic measurement.

For enzymatic digestion, myoblasts were differentiated on four well plates (as described in Example 5) and treated with either collagenase digestion solution or trypsin digestion solution. Therefore, supernatant of cultured cells was discarded and 300 μl of chosen enzyme-solution added. Collagenase and Trypsin digestion solutions had to be prepared preliminarily and contain ingredients as following:

Collagenase Digestion Solution:
98.43% (v/v) Ringerlactat (1, 8 mM)
0.13% (v/v) CaC12 (150 mM)
1.4% (v/v) Collagenase I

Trypsin Digestion Solution:
10× Trypsin solution diluted in PBS 1:10 to a resulting 1× Trypsin solution

Cells then have been incubated at 37° C. and 5% CO2 for two hours. Due to the digest, cells lost connection to surface, could be resuspended and put into a centrifugation tube. Next, cells have been centrifuged at 800 g for 10 min. Acetylcholinesterase activity was then measured either of the supernatant or the pellet. Therefore, 300 μl of supernatant was taken and 3 mg of AChE activity reagent added. For measurement of Pellet, 3 mg AChE activity reagent was solubilised in 300 μl buffer (either Assay Buffer or PBS). OD412 nm could then be measured as described above.

### Removal of Membrane-Bound AChE by Enzymatic Digestion

For enzymatic digestion, myoblasts were differentiated on four well plates (as described above) and treated with either collagenase digestion solution or trypsin digestion solution. Therefore, supernatant of cultured cells was discarded and 300 μl of chosen enzyme-solution added. Collagenase and Trypsin digestion solutions had to be prepared preliminarily and contain ingredients as following:

Collagenase Digestion Solution:
98. 43% (v/v) Ringerlactat (1, 8 mM)
0.13% (v/v) CaC12 (150 mM)
1. 4% (v/v) Collagenase I

Trypsin Digestion Solution:
10× Trypsin solution diluted in PBS 1:10 to a resulting 1× Trypsin solution.

Cells then have been incubated at 37° C. and 5% CO2 for two hours. Due to the digest, cells lost connection to surface, could be resuspended and put into a centrifugation tube. Next, cells have been centrifuged at 800 g for 10 min. acetylcholinesterase activity was then measured either of the supernatant or the pellet. Therefore, 300 μl of supernatant was taken and 3 mg of AChE activity reagent added. For measurement of Pellet, 3 mg AChE activity reagent was solubilised in 300 μl buffer (either Assay Buffer or PBS). OD412 nm could then be measured as described above.

### EXAMPLE 10

### Separation of Myogenic Progenitors

Thus cultivated cultures of skeletal muscle derived cells do not all have myogenic potential, myogenic progenitors were separated by MACS® separation technique. CD56 (NCAM1) expression of cultivated SMDC has been measured by FACS analysis (as described in Example 6 before and after running MACS column (as described in Example 7). It was observed that cultivated SMDCs have a CD56 positive as well as CD56 negative cell population. These populations could be separated by MACS® to a CD56 positive and CD56 negative subpopulation. The CD56 positive subpopulation comprised about 98% CD56 positive SMDC while the CD56 negative subpopulation comprised about 98% CD56 negative skeletal muscle derived non-myogenic cells.

### EXAMPLE 11

### Onset of Functional AChE Expression During Myoblast Differentiation

The onset of functional AChE expression during differentiation of mononucleated myoblasts to multinucleated myotubes has been assessed quantitatively as well as compared to non-myogenic cells in vitro. AChE activity increases during differentiation of myoblasts and seems to climax after several days. Therefore, AChE activity was calculated as described in Example 6. Thus on each day of AChE activity measurement a photo was taken it is visible that myoblasts begin to align on the first day of differentiation and myotubes appear on the third day. Therefore, it becomes clear that AChE activity, which shows to have its most increase during the second day of differentiation, happens at the stage of mononucleated myoblasts, which are in contact to each other but did not fuse already. Further experiments were carried out as described in Example 9. Therefore, AChE activity of differentiating myoblasts was tested during differentiation either in a cell-membrane permeable PBS buffer or in non-permeable Assay Buffer. It was observed that AChE activity increases during differentiation and that activity in Assay Buffer is higher than in PBS but shows related trend during differentiation.

### EXAMPLE 12

### Influence of Cell Count on AChE Activity

To identify the role of the cell count in relation to the overall AChE activity, different cell numbers of differentiating myoblasts were tested. CD56 positive Myoblasts (95.03%) have been differentiated for 6 days and afterwards AChE activity tested on a plate reader (as described in Example 9). It was observed that there is a linear regression between the cell number and the determined AChE activity.

### EXAMPLE 13

### Influence of Purity Level of Myogenic Progenitors on AChE Activity

Difference of AChE activity between skeletal muscle myogenic progenitor cells (CD 56 positive) and skeletal muscle derived non-myogenic cells was measured. As both cell types were extracted from the same skeletal muscle, MACS (as described in Example 7) was used to separate CD56 positive and CD56 negative cells. Mixtures of these myogenic progenitors and non-myogenic progenitors were created, differentiated for several days and AChE activity detected by Acetylcholinesterase assay (as described in Example 9) before and after differentiation. For visible correlation of CD56 and desmin expression pattern in SMDCs, mixtures of CD56 positive and negative cells were prepared. It was observed that the amount of desmin positive SMDCs increase analogue with the amount of CD56 positive SMDCs in culture. CD56 negative SMDCs were also desmin negative. Dates of this experiment were then analysed in order to calculate the AChE activity in units per millilitre. It was observed that AChE activity is proportional to purity of myogenic progenitor cells (CD56 positive and desmin positive) tested.

### EXAMPLE 14

### Influence of Trypsin and Collagenase on Membrane Bound AChE

To detect the effect of proteolytic enzymes on the membrane bound AChE, digestion of multinucleated myotubes with collagenase or trypsin was carried out as described above. Therefore, AChE activity of either the digested cells or the supernatant was measured. It was shown that AChE activity of multinucleated myotubes (measured in non-membrane permeable buffer, PBS) decreased after 2 hours of collagenase digestion. Also trypsin digestion seems to have an impact on membrane bound AChE because AChE activity of digestion solution increased after incubation with myotubes for 2 hours.

### EXAMPLE 15

### Potency Assay

To increase the attachment of cells in 96 well plates said 96 well plates were coated with fibronectin by incubating each 100 μl fibronectin (5 μg/ml) in the 96 wells for at least 40 minutes at 37° C., 5% CO2. Subsequently, the wells were washed once with 10×PBS.

SMDCS isolated from a biceps biopsy of a patient have been analysed by FACS analysis for the expression of CD56. Afterwards, 50000 CD56+ cells were pipetted in each well of two 96 well plates (plate 1 and 2). In a third 96 well plate (plate 3) 50000 cells were pipetted in each well of said 96 well plate, wherein said cells were composed of 60% CD56+ cells and 40% CD56− cells. Subsequently, each 200 μl Myoblast medium was added and the 96 well plates were incubated at 37° C., 5% CO2 overnight. On the next day the myoblast medium of plate 1 and 3 was aspirated. The wells were washed once with differentiation medium. Subsequently, 200 μl differentiation medium was added to each well and the plates were incubated at 37° C., 5% CO2 for five days. The AChE activity of plate 2 was measured at the same day on which differentiation medium was added to plates 1 and 3. The AChE activity of plates 1 and 3 was measured after incubation and thus 5 days after the addition of differentiation medium. For measuring the AChE activity of plate 2 the myoblast medium was aspirated and the 96 well plate was washed once with PBS. Afterwards, 100 μl of substrate solution (1 mg substrate per 100 μl PBS) was added to each well. Subsequently, the OD was measured at 412 nm in a Plate Reader (Anthos 2010) each 60 seconds for in total 60 minutes. For measuring the AChE activity of plates 1 and 3 five days after the measurement of AChE activity of plate 2 the differentiation medium is aspirated. Then, 100 μl substrate medium was added directly into the wells of the 96 well plates. The further steps were the same as performed for the measurement of the AChE activity of plate 2.

For evaluating the obtained data the changes of the OD between 60 minutes and the start of each single measurement is calculated and called “OD-change”. The OD-change of plates 1 and 3 and plate 2 are then divided for calculating the multiplication of the AChE activity during five days of differentiation.

The potency assay as described above have been performed for three different orders (FAUs) obtained from three different patients. The amount of CD56+ cells in each of the FAUs was as follows: Fau0114516 (96.57% CD56 positive), Fau0114523 (96.715% CD56 positive) and Fau0114517 (75% CD56 positive). The results are shown in the following table:

| | | OD Change | | multiplication |
|---|---|---|---|---|
| % CD56+ | sample | Start | 5 days Diff | 0-5 days Diff |
| 96.57 | Fau0114516 | 0.06 | 0.41 | 6.75 |
| 96.72 | Fau0114523 | 0.06 | 0.66 | 11.07 |
| 75.8 | Fau0114517 | 0.08 | 0.34 | 4.28 |
| 60 | Fau0114516 | 0.05 | 0.16 | 3.28 |
| 60 | Fau0114523 | 0.09 | 0.35 | 3.83 |
| 60 | Fau0114517 | 0.07 | 0.24 | 3.47 |

Thus, the multiplication of the AChE activity increases with the proportion of fusion competent CD56+ cells. Moreover, it was shown that the AChE activity of the cells not mixed with CD56− cells is in all cases higher than the AChE activity of the mixtures comprising 60% CD56+ cells.

### EXAMPLE 16

### Linearities of the Potency Assay

For proving the linearity of the potency assay mixtures of fusion competent CD56+ cells and CD56− cells, both obtained from a biceps biopsy of a patient, were prepared with ratios of 100%, 80%, 60% and 0% of CD56+ cells. The potency assay was performed as described in Example 15. The results of three different tests are shown in the following tables:

| | | OD | | |
|---|---|---|---|---|
| | | Change | | multiplication |
| Fau | % CD56⁺ | 0 days Diff | 5 days Diff | 0-5 days Diff |
| 114502 | 100 | 0.04 | 0.19 | 4.87 |
| 114511 | 100 | 0.03 | 0.67 | 19.79 |
| 114513 | 100 | 0.02 | 0.38 | 18.14 |
| 114502 | 80 | 0.04 | 0.16 | 3.88 |
| 114511 | 80 | 0.04 | 0.38 | 10.94 |
| 114513 | 80 | 0.03 | 0.18 | 6.13 |
| 114502 | 60 | 0.03 | 0.14 | 4.57 |
| 114511 | 60 | 0.05 | 0.33 | 7.22 |
| 114513 | 60 | 0.04 | 0.16 | 4.08 |
| CD56⁻ | 0 | 0.04 | 0.05 | 1.23 |
| blank value | n/a | 0.01 | 0.01 | 0.71 |

Mean value of AChE activity
multiplication in 5 days

| 100% | 80% | 60% | 0% |
|---|---|---|---|
| 14.27 | 6.99 | 5.29 | 1.23 |

Standard abbrevation

| | | | |
|---|---|---|---|
| 8.18 | 3.61 | 1.69 | — |

As shown in the table the multiplication of AChE activity increases with the proportion of CD56+ cells.

### EXAMPLE 17

### AChE Standard

A total of 8 AChE standard enzyme dilutions were prepared with the highest enzyme concentration of 500 mU/mL and the lowest was 4 mU/mL. All dilutions were prepared in 0.14 M phosphate buffer with 0.1% triton X-100, pH 7.2, and were tested in duplicate. A blank reaction was also included, which was composed of all reagents except AChE enzyme. 200 μL of each AChE enzyme dilution was placed in a 24-well plate. 300 μL 0.5 mM DTNB (prepared in 0.14 M phosphate buffer with 0.1% triton-X 100 at a pH of 7,2 were added to each enzyme dilution. Afterwards 50 μL of 5.76 mM Acetylthiocholine iodide (ATI) (prepared in distill water) were added. Measurement was performed for 15 minutes (15 cycles) in a plate reader at 412 nM and 30° C. Corrected 0D₄₁₂ values were obtained by subtracting blank measurement from mean OD₄₁₂. Standard curve for AChE was developed in GraphPad Prism 5 software by employing ‘non-linear regression’ followed by straight line equation, which resulted in an excellent R² (coefficient of determination) value of 0.9980. The straight line equation [1] for AChE was obtained as follows,

Y=0.003282X−0.01372 [1]

The AChE activity of any unknown sample (X) can be determined by equation [2], which is derived by equation [1] as follows,

$$X = \frac{Y + 0.01372}{0.003282} \qquad [ 2 ]$$

whereas, Y represents the OD₄₁₂ of any unknown sample subjected to AChE potency assay.

### EXAMPLE 18

### AChE Potency Assay

Four different classes of CD56⁺ fractions containing 2%, 60%, 80% and more than 80% CD56+ cells were prepared as follows: SMDC cells isolated from a patient were separated by Magnetic-activated cell sorting (MACS®) into CD56− cells and CD56+ cells. CD56 MicroBeads (human) kit was purchased from Miltenyi Biotec GmbH, Bergisch Gladbach, Germany for isolation of CD56− cells from aSMDCs. The separation was performed according to manufacturer's instructions. In summary, after isolating the MSDC from a patient's sample and measuring of entire cell number cells were centrifuged at 1300 rpm (400×g) for ten minutes, supernatant discarded and resuspended in 10 ml MACS-Buffer. After another centrifugation step (1300 rpm, 10 min) and discarding of supernatant, the pellet was resupended in 80 μl MACS-Buffer, each 10⁷ cells but not less than 80 μl. Subsequently 20 μl of magnetic CD56 antibody was added per 10⁷ cells and incubated for 15 minutes at 4° C. Subsequently, sorting of cells was carried out as described in MACS protocol with Mini MACS Separator and MS-column.

Afterwards, four different cell classes (Class 1 to Class 4) were generated, which were categorized according to their CD56+ cell population (corresponds to the myoblast fraction of total cell population) as mentioned in the following table. These four classes of cell types were generated to obtain a cut-off value of AChE enzyme units in a cell population with 60% CD56+ fraction.

| Classes of cells according | | |
|---|---|---|
| to their percentage of | | |
| CD56+ fraction Cell class | CD56+ % | CD56− % |
| Class 1 | >80 | <20 |
| Class 2 | 80 | 20 |
| Class 3 | 60 | 40 |
| Class 4 | 2 | 98 |

Class 1 and Class 4 (CD56− and CD56+ fraction isolated from SMDC by MACS as described above) cells were used without any mixing with CD56− fraction. Class 1 (>80% CD56+) and Class 4 (98% CD56−) cells were mixed in order to get Class 2 and Class 3 with 80% and 60% CD56+ fractions, respectively. A total of 120,000 cells were seeded for each class in a 24-well plate together with myomedium (Skeletal Muscle Cell Differentiation Medium—manufacturer: PromoCell). After 2 days of incubation at 37° C. the SMDCs were washed with 1×PBS and the medium was exchanged by differentiation medium (see Example 5). After further 6 days AChE activity was measured by a microplate reader at 412 nm. For doing this medium was aspirated from 24-well plate and 300 μL 0.5 MM 5,5′-Dithiobis(2-nitrobenzoic acid) (DTNB) (prepared in 0.14 M phosphate buffer with 0.1% triton-X 100, pH 7,2) was added to each well. After an incubation for 2 minutes at 30° C. 50 μL 5.76 mM ATI (prepared in distil water) was added. After further 10 minutes incubation at 30° C. AChE activity was measured for 50 minutes (50 cycles) in a Anthos Zeneyth 340rt microplate reader (Zenyth 340rt microplate reader user's manual, 2010) at 412 nm at 30° C. The data obtained were evaluated relative to AChE standard curve.

### EXAMPLE 19

### AChE Activity in Patients with Stress Urinary Incontinence

A total of 101 patient samples were subjected to AChE potency assay. Each sample was categorized into 4 classes (Class 1, Class 2, Class 3 and Class 4 with CD56% as >80, 80, 60 and 2) as described in Example 18. AChE activity was calculated in mUᵣₑₗ (relative milli-units) with reference to the straight line equation [1] for AChE standard as described in Example 17. The term ‘relative milliUnit’ was used because of the fact that AChE activity in standard dilutions were measured per mL at 15 min, whereas in patient samples AChE activity was determined per 120,000 aSMDCs in a 24-well plate at 50 min. The AChE activity of all CD56+ samples (Class 1, 2 and 3) was ranged from 60 mUᵣₑₗ to 452 mUᵣₑₗ signifying the highly variable AChE expression among different patients. The results are summarized in the following table.

| | >80% | 80% | 60% | 2% |
|---|---|---|---|---|
| | (Class 1) | (Class 2) | (Class 3) | (Class 4) |
| Mean AChE | 172.39 | 170.09 | 138.69 | 15.42 |
| activity [mUᵣₑₗ] | | | | |
| SD | 82.66 | 81.15 | 70.70 | 6.46 |
| SEM | 8.18 | 8.03 | 7.00 | 0.64 |

It was observed that within the same patient, CD56+ classes varied prominently e.g. one patient showed highest AChE activity (452 mUrel) at 80% CD56+ in comparison to >80% CD56+(290 mUrel) and even the 60% CD56+ (389 mUrel) outperformed >80% CD56+ fraction. The possible reason for this trend is the loss of big myotubes formed in 24-well plate during aspiration of medium at post 6 days in differentiation medium. The present data for AChE activity in 101 patients strongly suggest a reference range in compliance for QA testing to be set at >60 mUrel. This reference range clearly marks a boundary between a cell fraction with CD56+ and CD56− expression. The average AChE activity in CD56− fraction is 15.42±0.64 mUrel which is significantly lower than 60% CD56+ fraction, which was found to be 138.69±7.00 mUrel (***p<0.001 60% CD56+ vs. 2% CD56+).

Moreover, the AChE potency (activity) data from 101 patients were divided into 6 categories as shown in the following table

| Category | AChE (mUᵣₑₗ) |
|---|---|
| 1 | ≧60-70 |
| 2 | 71-80 |
| 3 | 81-90 |
| 4 | 90-100 |
| 5 | >100 |
| 6 | >200 |

By this categorization it could be proved that the AChE activity was directly proportional to the number of CD56⁺ cells seeded in 24-well plate. For example the number of patients having >200 mUᵣₑₗ AChE activity (category 6) with 60% CD56⁺ cell fraction was nearly double the number compared to 80% or >80% . AChE proportionality with CD56⁺ fraction was confirmed by the observation of category 1 (≧60-70 mUᵣₑₗ) as well.

1. A potency assay for skeletal muscle derived cells (SMDC), the potency assay comprising the steps of:
(a) measuring AChE activity of SMDC, and
(b) evaluating the potential of the SMDC to be used for the treatment of skeletal muscle dysfunction based on the AChE activity measured in step (a).

2. The potency assay according to claim 1, wherein the SMDC of step (a) are CD56 positive and/or desmin positive cells.

3. The potency assay according to claim 1, wherein the SMDC have the potential to be used for the treatment of skeletal muscle dysfunction, if the AChE activity of the SMDC is at least twice as high as the AChE activity of non-myogenic cells.

4. The potency assay according to claim 3, wherein the non-myogenic cells are CD56 negative and/or desmin negative cells.

5. The potency assay according to claim 1, wherein the potency assay comprises the steps of:
(a) incubating skeletal muscle derived cells in a cell growth medium,
(b) incubating the cells obtained in step a) with a differentiation medium,
(c) detecting AChE activity at two or more different points in time wherein the first detection of the AChE activity is performed on the starting day of step (b), and
(d) comparing the AChE activity at the two or more different points in time, wherein the difference between the AChE activities at the two different points in time indicates the potency of said skeletal muscle derived cells.

6. The potency assay according to claim 1, wherein the SMDC of step (a) comprise at least 60% skeletal muscle derived cells expressing CD56 and/or desmin.

7. The potency assay according to claim 5, wherein the time difference between the two different points in time is 3, 4, 5, 6 or 7 days.

8. The potency assay according to claim 1, wherein the skeletal muscle dysfunction is incontinence, in particular a urinary and/or an anal incontinence.

9. A skeletal muscle derived cell (SMDC) composition comprising at least 60% CD56 positive and/or desmin positive cells, wherein the AChE activity of said SMDC is at least twice as high as the AChE activity of non-myogenic cells.

10. The SMDC composition according to claim 9, wherein said SMDC are identifiable by a potency assay comprising the steps of:
(a) measuring AChE activity of SMDC, and
(b) evaluating the potential of the SMDC to be used for the treatment of skeletal muscle dysfunction based on the AChE activity measured in step (a).

11. The SMDC composition according to claim 9, wherein said SMDC are 60% CD56 positive and desmin positive cells.

12. The SMDC composition according to claim 9, wherein said SMDC are primary cells.

13. A method of treating skeletal muscle dysfunction comprising administering to said subject skeletal muscle derived cell (SMDC) composition comprising at least 60% CD56 positive and/or desmin positive cells, wherein the AChE activity of said SMDC is at least twice as high as the AChE activity of non-myogenic cells.

14. The method of claim 13, wherein the muscle dysfunction is incontience.

15. A kit comprising (a) an agent or agents that assess CD56 expression, (b) an agent or agents that assess desmin expression, and (c) an agent or agents that assess AChE activity.

16. The potency assay according to claim 6, wherein the time difference between the two different points in time is 3, 4, 5, 6 or 7 days.

17. The method of claim 14, wherein the incontinence is urinary and/or anal incontinence.
